(19) United States
(12) Patent Application Publication
(10) Pub. No.: US 2016/0038929 A1
(43) Pub. Date: Feb. 11, 2016

(54) Regeneration of Aromatic Alkylation Catalyst Using Ozone

(71) Applicant: ExxonMobil Chemical Patents Inc., Baytown, TX (US)

(72) Inventors: Ranier Kolb, Kingwood, TX (US); Terry E. Helton, Montgomery, TX (US); Allen W. Burton, Stewartsville, NJ (US); Karl G. Strohmaier, Port Murray, NJ (US); Matthew J. Vincent, Kingwood, TX (US); Chunshe J. Cao, Conroe, TX (US); Dominick A. Zurlo, Easton, PA (US)

(21) Appl. No.: 14/781,769

(22) PCT Filed: Apr. 22, 2014
(86) PCT No.: PCT/US2014/034994

§ 371 Date: Oct. 1, 2015

Related U.S. Application Data
(60) Provisional application No. 61/821,589, filed on May 9, 2013.

(30) Foreign Application Priority Data
Jul. 22, 2013 (EP) 13177336.8

(51) Int. Cl. B01J 38/04; B01J 29/70; C07C 2/66

(52) U.S. Cl. CPC B01J 38/04; C07C 2/66; B01J 29/7007; B01J 29/7038; B01J 29/70; C07C 2529/70

(57) ABSTRACT

The present disclosure relates to a method of regenerating an at least partially deactivated catalyst, preferably an aromatic alkylation or transalkylation catalyst, comprising a molecular sieve. The method comprises the step of contacting the deactivated catalyst with an ozone-containing gas, preferably at a temperature of about 50° C. to about 250° C.

### PRIORITY CLAIM

This application claims the benefits of U.S. Provisional Application Ser. No. 61/821,589 filed May 9, 2013, and claims priority to EP 13177336.8 filed Jul. 22, 2013, which are incorporated by reference in their entireties.

### FIELD OF THE INVENTION

The present invention relates to a method of regenerating an at least partially deactivated catalyst, preferably a deactivated alkylation catalyst or a deactivated transalkylation catalyst, and a process for producing mono-alkylaromatic compounds with a regenerated catalyst.

### BACKGROUND OF THE INVENTION

Mono-alkylaromatic compounds, such as ethylbenzene and cumene, are valuable commodity chemicals which are used industrially for the production of styrene monomer and phenol respectively. Ethylbenzene may be produced by a number of different chemical processes, but one process which has achieved a significant degree of commercial success is the vapor phase alkylation of benzene with ethylene in the presence of a solid, acidic ZSM-5 zeolite catalyst. In the commercial operation of this process, the poly-alkylated benzenes, including both polymethylated and polyethylated benzenes, which are inherently co-produced with ethylbenzene in the alkylation reactor, are transalkylated with benzene to produce additional ethylbenzene either by being recycled to the alkylation reactor or by being fed to a separate transalkylation reactor. Examples of such ethylbenzene production processes are described in U.S. Pat. Nos. 3,751,504 (Keown), 4,547,605 (Kresge), and 4,016,218 (Haag).

More recent focus has been directed at liquid phase processes for producing ethylbenzene from benzene and ethylene, since liquid phase processes operate at a lower temperature than their vapor phase counterparts and hence tend to result in lower yields of by-products. For example, U.S. Pat. No. 4,891,458 describes the liquid phase synthesis of ethylbenzene with zeolite beta, whereas U.S. Pat. No. 5,334,795 describes the use of MCM-22 in the liquid phase synthesis of ethylbenzene.

Cumene has for many years been produced commercially by the liquid phase alkylation of benzene with propylene over a Friedel-Craft catalyst, particularly solid phosphoric acid or aluminum chloride. More recently, however, zeolite-based catalyst systems have been found to be more active and selective for propylation of benzene to cumene. For example, U.S. Pat. No. 4,992,606 describes the use of MCM-22 in the liquid phase alkylation of benzene with propylene.

Other molecular sieves known for use as liquid phase alkylation and transalkylation catalysts include MCM-36 (see U.S. Pat. No. 5,258,565), MCM-49 (see U.S. Pat. No. 5,371,310) and MCM-56 (see U.S. Pat. No. 5,453,554).

In catalytic processes, the catalyst deactivates with time on stream and needs to be regenerated to recover activity. Typically, zeolite catalysts are regenerated by flowing air to burn off coke at high temperature and remove other deactivating species. However the burning of the deactivated catalyst usually needs be conducted at a high temperature. Many methods for regeneration of the deactivated alkylation or transalkylation catalysts have been developed recently.

U.S. Pat. No. 7,037,781 B1 discloses a process for regenerating a hydrocarbon conversion catalyst comprising zeolite L with ozone. The catalyst is contacted with ozone at a temperature of from about 20° C. to about 250° C. and a concentration of ozone of from about 0.1 to about 5 mole percent. The process is particularly useful for reforming and dehydrocyclodimerization catalysts.

Some studies on ozone-related regeneration have been made, for example, “Influence of O₂ and O₃ Regeneration on the Metallic Phase of the Pt—Re/Al₂O₃ Catalyst” by C. L. Pieck et al., Applied Catalysis A: General 165 (1997), pp. 207-218; “Isobutane/butane Alkylation: Regeneration of Solid Acid Catalyst” by Carlos A. Querini, Catalysis Today 62 (2000), pp. 135-143; the article “Regeneration of Pentasil Zeolite Catalysts Using Ozone and Oxygen” by R. G. Copperthwaite et al., J. Chem. Soc., Faraday Trans. 1, 1986, pp. 1007-1017; “Isobutane Alkylation with C₄ Olefins: Low Temperature Regeneration of Solid Acid Catalysts with Ozone Catalyst Deactivation 1997”, by C. A. Querini et. al., Proceedings of the 7th International Symposium, Cancun, Mexico, Oct. 5-8, 1997; Studies in Surface Science and Catalyst, Vol. 111, pp. 407-414, 1997, Elsevier Science B.V. “Differential Effect of Coke Burning With Oxygen or Ozone on Pt—Re Interaction on Pt—Ze/Al₂O₃ Catalyst Deactivation”, by C. L. Pieck et al., Proceedings of the 7th International Symposium, Cancun, Mexico, Oct. 5-8, 1997, Studies in Surface Science and Catalyst, Vol. 111, pp. 407-414, 1997, Elsevier Science B.V.; and “Regeneration of Coked Pt—Re/Al₂O₃ Catalyst by Burning with Oxygen and Ozone Catalyst Deactivation 1994”, by C. L. Pieck et al., Studies in Surface Science and Catalyst, Vol. 88, pp. 289-295, 1994 Elsevier Science B.V., The Netherlands.

Other regeneration processes can be found in U.S. Pat. Nos. 6,781,025, 6,909,026 and 6,911, 568, and European Patent No. 2217374.

According to the invention, it has now been found that contacting the at least partially deactivated catalyst comprising a molecular sieve with an ozone-containing gas is effective in restoring the activity and selectivity of the at least partially deactivated catalyst. This novel method of the present disclosure provides an efficient and convenient way for regeneration of deactivated catalysts and provides effective restoration of activity and selectivity of the catalyst.

### SUMMARY OF THE INVENTION

In one aspect, the invention resides in a method of regenerating an at least partially deactivated catalyst, for example, an aromatic alkylation or a transalkylation catalyst, comprising a molecular sieve; the method comprising the step of contacting the deactivated catalyst with an ozone-containing gas under regeneration conditions.

Preferably, the molecular sieve of the catalyst is selected from the group consisting of a MCM-22 family molecular sieve, faujasite, mordenite, zeolite beta, and combinations thereof. Preferably, the MCM-22 family molecular sieve is selected from the group consisting of MCM-22, PSH-3, SSZ-25, MCM-36, MCM-49, MCM-56, ERB-1, EMM-10, EMM-10-P, EMM-12, EMM-13, UZM-8, UZM-8HS, ITQ-1, ITQ-2, ITQ-30 and combinations thereof.

Preferably, the contacting step is conducted in-situ and at a temperature from about 50° C. to about 250° C., preferably for a period from 10 minutes to 48 hours in another embodiment, and preferably at a pressure of about 100 kPa to about 5000 kPa.

Preferably, the ozone-containing gas has the ozone concentration of from about 0.1 to 10.0 wt. %, and preferably has a flow rate of about 0.1 to about 900 volumes, or of from about 1 to about 900 volumes of ozone-containing gas to catalyst volume per minute under regeneration conditions.

In a further aspect, the present invention resides in a process for alkylating or transalkylating an alkylatable aromatic compound comprising the step of contacting the alkylatable aromatic compound and an alkylating agent with a regenerated catalyst, preferably a regenerated alkylation catalyst or a regenerated transalkylation catalyst, comprising a molecular sieve under alkylation conditions or transalkylation conditions to form an alkylated aromatic compound, wherein the regenerated catalyst was regenerated by a method comprising the step of contacting an at least partially deactivated catalyst with an ozone-containing gas under regeneration conditions.

Preferably, the molecular sieve of the catalyst is selected from the group consisting of a MCM-22 family molecular sieve, faujasite, mordenite, zeolite beta, and combinations thereof. Preferably, the MCM-22 family molecular sieve is selected from the group consisting of MCM-22, PSH-3, SSZ-25, MCM-36, MCM-49, MCM-56, ERB-1, EMM-10, EMM-10-P, EMM-12, EMM-13, UZM-8, UZM-8HS, ITQ-1, ITQ-2, ITQ-30 and combinations thereof.

Preferably, the alkylation conditions or the transalkylation conditions are such that the alkylatable aromatic compound and alkylating agent are in at least partial liquid phase conditions; preferably, liquid phase conditions.

Preferably, the alkylating agent includes an alkylating olefinic group having 1 to 5 carbon atoms, or a poly-alkylated aromatic compound.

Preferably, the alkylating agent is ethylene or propylene and preferably, the alkylatable aromatic compound is benzene.

Preferably, the alkylation conditions or the transalkylation conditions comprise a temperature of from about 50° C. to about 400° C. and a pressure of from about 100 kPa to about 7000 kPa.

### DETAILED DESCRIPTION OF THE INVENTION

The present invention relates to a process for the production of an alkylated aromatic compound, preferably, a mono-alkylaromatic compound, particularly ethylbenzene or cumene, by the at least partial liquid phase alkylation of an alkylatable aromatic compound with an alkylating agent in the presence of regenerated catalyst, for example, a regenerated alkylation catalyst or a regenerated transalkylation catalyst, comprising a molecular sieve. More particularly, the invention is concerned with a process in which the catalyst is regenerated via an in-situ catalyst regeneration step when such catalyst has become at least partially deactivated. In the catalyst regeneration step, the at least partially deactivated catalyst is contacted with an ozone-containing gas at a temperature of about 50° C. to about 250° C. so as to reactivate the catalyst substantially without loss of its mono-alkylation selectivity.

The term “alkylatable aromatic compound” as used herein means an aromatic compound that may receive an alkyl group. One non-limiting example of an alkylatable aromatic compound is benzene.

The term “alkylating agent” as used herein means a compound which may donate an alkyl group to an alkylatable aromatic compound. Non-limiting examples of an alkylating agent are ethylene, propylene, and butylene. Another non-limiting example is any poly-alkylated aromatic compound that is capable of donating an alkyl group to an alkylatable aromatic compound.

The term “aromatic” as used herein in reference to the alkylatable aromatic compounds which are useful herein is to be understood in accordance with its art-recognized scope which includes substituted and unsubstituted mono- and polynuclear compounds. Compounds of an aromatic character which possess a heteroatom (e.g., N or S) are also useful provided they do not act as catalyst poisons, as defined below, under the reaction conditions selected.

The term “liquid phase” as used herein means a mixture having at least 1 wt. % liquid phase, optionally at least 5 wt. % liquid phase, at a given temperature, pressure, and composition.

The term “at least partially deactivated”, or “deactivated”, as used herein means alkylation or transalkylation activity of the catalyst is decreased by an amount of at least 1% deactivated compared to initial alkylation activity of the catalyst.

The term “framework type” is used herein has the meaning described in the “Atlas of Zeolite Framework Types,” by Ch. Baerlocher, W. M. Meier and D. H. Olson (Elsevier, 5th Ed., 2001).

The term “MCM-22 family material” (or “MCM-22 family molecular sieve”), as used herein, can include:
(i) molecular sieves made from a common first degree crystalline building block “unit cell having the MWW framework topology.” A unit cell is a spatial arrangement of atoms which is tiled in three-dimensional space to describe the crystal as described in the “Atlas of Zeolite Framework Types,” by Ch. Baerlocher, W. M. Meier and D. H. Olson (Elsevier, 5th Ed., 2001);
(ii) molecular sieves made from a common second degree building block, a 2-dimensional tiling of such MWW framework type unit cells, forming a “monolayer of one unit cell thickness,” preferably one c-unit cell thickness;
(iii) molecular sieves made from common second degree building blocks, “layers of one or more than one unit cell thickness”, wherein the layer of more than one unit cell thickness is made from stacking, packing, or binding at least two monolayers of one unit cell thick of unit cells having the MWW framework topology. The stacking of such second degree building blocks can be in a regular fashion, an irregular fashion, a random fashion, and any combination thereof; or
(iv) molecular sieves made by any regular or random 2-dimensional or 3-dimensional combination of unit cells having the MWW framework topology.

The MCM-22 family materials are characterized by having an X-ray diffraction pattern including d-spacing maxima at 12.4±0.25, 3.57±0.07 and 3.42±0.07 Angstroms (either calcined or as-synthesized). The MCM-22 family materials may also be characterized by having an X-ray diffraction pattern including d-spacing maxima at 12.4±0.25, 6.9±0.15, 3.57±0.07 and 3.42±0.07 Angstroms (either calcined or as-synthesized). The X-ray diffraction data used to characterize the molecular sieve are obtained by standard techniques using the K-alpha doublet of copper as the incident radiation and a diffractometer equipped with a scintillation counter and associated computer as the collection system.

The term “mono-alkylaromatic compound” means an aromatic compound that has only one alkyl substituent. Non-limiting examples of mono-alkylaromatic compounds are ethylbenzene, iso-propylbenzene (cumene) and sec-butylbenzene.

The term “poly-alkylaromatic compound” as used herein means an aromatic compound that has more than one alkyl substituent. A non-limiting example of a poly-alkylaromatic compound is poly-alkylated benzene, e.g., di-ethylbenzene, tri-ethylbenzene, di-isopropylbenzene, and tri-isopropylbenzene.

Substituted alkylatable aromatic compounds which can be alkylated herein must possess at least one hydrogen atom directly bonded to the aromatic nucleus. The aromatic rings can be substituted with one or more alkyl, aryl, alkaryl, alkoxy, aryloxy, cycloalkyl, halide, and/or other groups which do not interfere with the alkylation reaction.

Suitable alkylatable aromatic hydrocarbons include benzene, naphthalene, anthracene, naphthacene, perylene, coronene, and phenanthrene, with benzene being preferred.

Generally the alkyl groups which can be present as substituents on the aromatic compound contain from 1 to about 22 carbon atoms and usually from about 1 to 8 carbon atoms, and most usually from about 1 to 4 carbon atoms.

Suitable alkyl substituted aromatic compounds include toluene, xylene, isopropylbenzene, normal propylbenzene, alpha-methylnaphthalene, ethylbenzene, cumene, mesitylene, durene, p-cymene, butylbenzene, pseudocumene, o-diethylbenzene, m-diethylbenzene, p-diethylbenzene, isoamylbenzene, isohexylbenzene, pentaethylbenzene, pentamethylbenzene; 1,2,3,4-tetraethylbenzene; 1,2,3,5-tetramethylbenzene; 1,2,4-triethylbenzene; 1,2,3-trimethylbenzene, m-butyltoluene; p-butyltoluene; 3,5-diethyltoluene; o-ethyltoluene; p-ethyltoluene; m-propyltoluene; 4-ethyl-m-xylene; dimethylnaphthalene; ethylnaphthalene; 2,3 -dimethylanthracene; 9-ethylanthracene; 2-methylanthracene; o-methylanthracene; 9,10-dimethylphenanthrene; and 3-methyl-phenanthrene. Higher molecular weight alkylaromatic hydrocarbons can also be used as starting materials and include aromatic hydrocarbons such as are produced by the alkylation of aromatic hydrocarbons with olefin oligomers. Such products are frequently referred to in the art as alkylate and include hexylbenzene, nonylbenzene, dodecylbenzene, pentadecylbenzene, hexyltoluene, nonyltoluene, dodecyltoluene, pentadecyltoluene, etc. Very often alkylate is obtained as a high boiling fraction in which the alkyl group attached to the aromatic nucleus varies in size from about C₆ to about C₁₂. When cumene or ethylbenzene is the desired product, the present process produces acceptably little by-products such as xylenes. The xylenes made in such instances may be less than about 500 ppm.

Reformate containing substantial quantities of benzene, toluene and/or xylene constitutes a particularly useful feed for the alkylation process of this invention.

The alkylating agents which are useful in the process of this invention generally include any aliphatic or aromatic organic compound having one or more available alkylating olefinic groups capable of reaction with the alkylatable aromatic compound, preferably with the alkylating group possessing from 1 to 5 carbon atoms. Non-limiting examples of suitable alkylating agents are olefins such as ethylene, propylene, the butenes, and the pentenes; alcohols (inclusive of monoalcohols, dialcohols, trialcohols, etc.) such as methanol, ethanol, the propanols, the butanols, and the pentanols; aldehydes such as formaldehyde, acetaldehyde, propionaldehyde, butyraldehyde, and n-valeraldehyde; and alkyl halides such as methyl chloride, ethyl chloride, the propyl chlorides, the butyl chlorides, and the pentyl chlorides, and so forth. After non-limiting examples of alkylating agents are the poly-alkylaromatic compounds.

Mixtures of light olefins are especially useful as alkylating agents in the alkylation process of this invention. Accordingly, mixtures of ethylene, propylene, butenes, and/or pentenes which are major constituents of a variety of refinery streams, e.g., fuel gas, gas plant off-gas containing ethylene, propylene, etc., naphtha cracker off-gas containing light olefins, refinery FCC propane/propylene streams, etc., are useful alkylating agents herein. For example, a typical FCC light olefin stream possesses the following composition:

| | Wt. % | Mol. % |
|---|---|---|
| Ethane | 3.3 | 5.1 |
| Ethylene | 0.7 | 1.2 |
| Propane | 14.5 | 15.3 |
| Propylene | 42.5 | 46.8 |
| Isobutane | 12.9 | 10.3 |
| n-butane | 3.3 | 2.6 |
| Butenes | 22.1 | 18.32 |
| Pentanes | 0.7 | 0.4 |

Reaction products which may be obtained from the process of the invention include ethylbenzene from the reaction of benzene with ethylene, cumene from the reaction of benzene with propylene, ethyltoluene from the reaction of toluene with ethylene, cymenes from the reaction of toluene with propylene, and sec-butylbenzene from the reaction of benzene and n-butenes.

The alkylation process of this invention is conducted such that the organic reactants, i.e., the alkylatable aromatic compound and the alkylating agent, are brought into contact with an alkylation catalyst in a suitable reaction zone such as, for example, in a flow reactor containing a fixed bed of the catalyst composition, under effective alkylation conditions or transalkylation conditions. Such conditions can include at least one of the following: a temperature of from about 50° C. and about 400° C., preferably from about 70° C. to about 300° C., a pressure of from about 100 kPa to about 7000 kPa, preferably from about 300 kPa to about 5000 kPa, a molar ratio of alkylatable aromatic compound to alkylating agent of from about 0.1:1 to about 50:1, preferably from about 0.5:1 to 10:1, and a feed weight hourly space velocity (WHSV) of between about 0.1 and 100 hr⁻¹, preferably from about 0.5 to 50 hr⁻¹.

The reactants can be in either the vapor phase or the liquid phase and can be neat, i.e., free from intentional admixture or dilution with other material, or they can be brought into contact with the zeolite catalyst composition with the aid of carrier gases or diluents such as, for example, hydrogen or nitrogen.

When benzene is alkylated with ethylene to produce ethylbenzene, the alkylation reaction may be carried out in the liquid phase. Suitable liquid phase conditions include a temperature between about 150° C. and 300° C., preferably between about 200° C. and 260° C., a pressure up to about 20000 kPa, preferably from about 200 kPa to about 5600 kPa, a WHSV of from about 0.1 hr⁻¹to about 50 hr⁻¹, preferably from about 1 hr⁻¹ and about 10 hr⁻¹ based on the ethylene feed, and a ratio of the benzene to the ethylene in the alkylation reactor from 1:1 to 30:1 molar, preferably from about 1:1 to 10:1 molar.

When benzene is alkylated with propylene to produce cumene, the reaction may also take place under liquid phase conditions including a temperature of up to about 250° C., preferably from about 10° C. to about 200° C.; a pressure up to about 25000 kKPa, preferably from about 100 kPa to about 3000 kPa; and a WHSV of from about 1 hr⁻¹ to about 250 hr⁻¹, preferably from 5 hr⁻¹ to 50 hr⁻¹, preferably from about 5 hr⁻¹ to about 10 hr⁻¹ based on the propylene feed.

In some embodiments, the alkylation catalyst comprises a MCM-22 family molecular sieve. The MCM-22 family molecular sieves have been found to be useful in alkylation and transalkylation processes for production of mono-alkylaromatic compounds. Examples of MCM-22 family molecular sieve are MCM-22 (described in U.S. Pat. No. 4,954,325), MCM-36 (described in U.S. Pat. No. 5,250,277), MCM-49 (described in U.S. Pat. No. 5,236,575), MCM-56 (described in U.S. Pat. No. 5,362,697), PSH-3 (described in U.S. Pat. No. 4,439,325), ITQ-1 (described in U.S. Pat. No. 6,077,498), ITQ-2 (described in International Patent Publication No. WO97/17290), ITQ-30 (described in International Patent Publication No. WO2005118476), SSZ-25 (described in U.S. Pat. No. 4,826,667), an EMM-10 family molecular sieve molecular sieve (described or characterized in U.S. Pat. Nos. 7,959,899 and 8,110,176, and U.S. Patent Application Publication No. 2008/0045768), such as EMM-10, EMM-12, EMM-13, ERB-1 (described in European Patent No. 0293032), UZM-8 (described in U.S. Pat. No. 6,756,030) and UZM-8HS (described in U.S. Pat. No. 7,713,513). A more preferred MCM-22 family molecular sieve can comprise

MCM-22, MCM-36, MCM-49 and MCM-56. In other embodiments, the alkylation catalyst can comprise faujasite, mordenite, and zeolite beta (described in detail in U.S. Pat. No. 3,308,069).

The molecular sieve can be combined in conventional manner with an oxide binder, such as alumina, such that the final alkylation catalyst contains between 2 and 80 wt. % sieve.

As the alkylation or transalkylation process of the invention proceeds, the catalyst will gradually lose its alkylation or transalkylation activity and selectivity because of carbonaceous and other materials adsorbed thereto, such that the reaction temperature required achieving a given performance parameter, for example, conversion of the alkylating agent will increase. According to the invention, when the activity of the catalyst has decreased by some predetermined amount, typically 5% to 90% and, more preferably 10% to 50%, compared to the initial activity of the catalyst, the deactivated catalyst is subjected to the novel regeneration method of the invention.

The regeneration method of the invention comprises the steps of contacting the deactivated catalyst with an ozone-containing gas under effective regeneration conditions, which may comprise at least one of the following conditions: a temperature of from about 50° C. to about 250° C., preferably from about 100° C. to about 220° C., more preferably from about 150° C. to about 200° C.; a period of from about 10 minutes to about 48 hours, preferably from about 10 minutes to about 24 hours, more preferably from about 30 minutes to about 12 hours; and a pressure of from about 100 kPa to about 5000 kPa, preferably from about 200 kPa to about 4000 kPa, more preferably from 300 kPa to about 3500 kPa.

The ozone-containing gas can have the ozone concentration of from about 0.1 wt. % to about 10 wt. %, preferably from about 0.5 wt. % to about 5 wt. %. Other gas components comprised in the ozone-containing gas may be any gas that is not reactive under the regeneration conditions, such as, air, nitrogen, oxygen, and an inert gas. The ozone-containing gas can have a flow rate of from about 1 to about 900 volumes of ozone-containing gas to catalyst volume per minute, preferably from about 10 to about 500 volumes of ozone-containing gas to catalyst volume per minute.

The ozone can be generated from oxygen, water, or air by any known method and/or generator. Non-limiting examples of ozone generator can include those commercially available from Ozone Solution Inc., Hull, Iowa, USA, for example, TG-series ozone generators. The ozone generation rate can be greater than 10 g/hr, or greater than about 50 g/hr, or greater than about 100 g/hr, or greater than about 150 g/hr, or greater than about 200 g/hr, for example, from about 200 g/hr to about 300 g/hr.

The regeneration method of the invention is found to be effective in restoring the activity and selectivity of the catalyst comparable to the parameters of catalyst regenerated by calcination at high temperature.

The alkylation process of the invention is particularly intended to produce mono-alkylaromatic compounds, such as ethylbenzene and cumene, but the alkylation step will normally produce some poly-alkylaromatic compounds. Thus, the process preferably includes the further steps of separating the poly-alkylaromatic compounds from the alkylation effluent and reacting them with additional aromatic feed in a transalkylation reactor over a suitable transalkylation catalyst. The transalkylation catalyst is preferably a molecular sieve which is selective to the production of the desired mono-alkylaromatic compound and can, for example, employ the same molecular sieve as the alkylation catalyst, such as MCM-22, MCM-49, MCM-56 and zeolite beta. In addition, the transalkylation catalyst may be faujasite and mordenite, such as TEA-mordenite.

The transalkylation reaction of the invention is conducted in the liquid phase under suitable conditions such that the poly-alkylaromatic compounds react with the additional aromatic feed (i.e., an alkylatable aromatic compound) to produce additional mono-alkylaromatic compound. Suitable transalkylation conditions include a temperature of 100° C. to 260° C., a pressure of about 200 kPa to about 600 kPa, a weight hourly space velocity of 1 to 10 on total feed, and aromatic feed/poly-alkylaromatic compound weight ratio 1:1 to 6:1.

When the poly-alkylaromatic compounds are polyethylbenzenes and are reacted with benzene to produce ethylbenzene, the transalkylation conditions preferably include a temperature of from about 220° C. to about 260° C., a pressure of from about 300 kPa to about 400 kPa, weight hourly space velocity of 2 to 6 on total feed and benzene/PEB weight ratio 2:1 to 6:1.

When the poly-alkylaromatic compounds are polyisopropylbenzenes (PIPB) and are reacted with benzene to produce cumene, the transalkylation conditions preferably include a temperature of from about 100° C. to about 200° C., a pressure of from about 300 kPa to about 400 kPa, a weight hourly space velocity of 1 to 10 on total feed and benzene/PIPB weight ratio 1:1 to 6:1.

As the transalkylation catalyst becomes deactivated, it may be subjected to the same regeneration process as described above in relation to the alkylation catalyst. Accordingly, the present invention also resides in a process for transalkylating an poly-alkylaromatic compound comprising the steps of:
(a) contacting an alkylatable aromatic compound and a poly-alkylaromatic compound with a transalkylation catalyst comprising a molecular sieve under transalkylation conditions to form a mono-alkylaromatic compound; and
(b) when the transalkylation catalyst has become at least partially deactivated, contacting the transalkylation catalyst with an ozone-containing gas under regeneration conditions.

The invention will now be more particularly described with reference to the following Examples. In the Examples, the activity and selectivity of a catalyst were measured based on benzene alkylation with propylene. Catalyst activity was calculated using the intrinsic second order rate constant for the formation of cumene under the reaction conditions (temperature 130° C. and pressure 2758 kPa). Reaction rate-constants were calculated using methods known to those skilled in the art. See “Principles and Practice of Heterogeneous Catalyst”, J. M. Thomas, W. J. Thomas, VCH, 1st Edition, 1997, the disclosure of which is incorporated herein by reference. Catalyst selectivity was calculated using the weight ratio of di-isopropyl benzenes produced to cumene produced (DIPB/IPB) and tri-isopropyl benzenes produced to cumene produced (Tri-IPB/IPB) under the reaction conditions (temperature 130° C. and pressure 2758 kPa).

### EXAMPLE 1

A catalyst comprising 80 wt. % MCM-49 (described in U.S. Pat. No. 5,236,575) and 20 wt. % Al₂O₃ deactivated in production of ethylbenzene by alkylation of benzene and ethylene was withdrawn. The deactivated catalyst (spent catalyst) was known to have carbonaceous and other material adsorbed thereto. The deactivated catalyst comprised carbons, sulfurs and other materials. One-half gram of the deactivated was charged to an isothermal well-mixed Parr autoclave reactor along with a mixture comprising of benzene (156 g) and propylene (28 g). The reaction was carried out at 130° C. and 2758 kPa for 4 hours. The catalyst performance was assessed and shown in Table 1.

### EXAMPLE 2

The deactivated catalyst of Example 1 was regenerated in bone dry air by calcining in an N₂/O₂ mixture at 538° C. for 6 hours. One-half gram of the regenerated catalyst was evaluated for benzene alkylation with propylene according to the method described in Example 1. The catalyst performance was assessed and shown in Table 1.

### EXAMPLE 3

The deactivated catalyst of Example 1 was regenerated in a flowing zone at 150° C. in a horizontal tube furnace for 16 hours using an ozone-containing gas having the ozone concentration of 1.2 wt. % and 98.2 wt. % air in a flow rate of 3500 sccm (standard cubic centimeter, 20° C., 1 atmosphere). One-half gram of the regenerated catalyst was evaluated for benzene alkylation with propylene according to the method described in Example 1. The catalyst performance was assessed and shown in Table 1.

### EXAMPLE 4

The deactivated catalyst of Example 1 was regenerated in a flowing zone at 200° C. in a horizontal tub furnace for 16 hours using an ozone-containing gas having the ozone concentration of 1.2 wt. % in a flow rate of 3500 sccm (standard cubic centimeter, 20° C., 1 atmosphere). One-half gram of the regenerated catalyst was evaluated for benzene alkylation with propylene according to the method described in Example 1. The catalyst performance was assessed and shown in Table 1.

### EXAMPLE 5

A catalyst comprising 65 wt. % MCM-22 (as described in U.S. Pat. No. 4,954,325) and 35 wt. % Al₂O₃ deactivated in production of cumene by alkylation of benzene and propylene was withdrawn. The deactivated catalyst was known to have carbonaceous material adsorbed thereto. The deactivated catalyst comprised carbons, sulfurs and other materials. One gram of the deactivated was charged to an isothermal well-mixed Parr autoclave reactor along with a mixture comprising of benzene (156 g) and propylene (28 g). The reaction was carried out at 130° C. and 2758 kPa for 4 hours. The catalyst performance was assessed and shown in Table 1.

### EXAMPLE 6

The deactivated catalyst of Example 5 was regenerated in a flowing zone at 150° C. in a horizontal tub furnace for 16 hours using an ozone-containing gas having the ozone concentration of 1.5 wt. % in a flow rate of 3500 sccm (standard cubic centimeter, 20° C., 1 atmosphere). One gram of the regenerated catalyst was evaluated for benzene alkylation with propylene according to the method described in Example 1. The catalyst performance was assessed and shown in Table 1.

### EXAMPLE 7

A catalyst comprising 80 wt. % zeolite beta (as described in U.S. Pat. No. 3,308,069) and 20 wt. % Al₂O₃ deactivated in production of ethylbenzene by alkylation of benzene and ethylene was withdrawn. The deactivated catalyst was known to have carbonaceous material adsorbed thereto. The deactivated catalyst comprised carbons, sulfurs and other materials. One-half gram of the deactivated was charged to an isothermal well-mixed Parr autoclave reactor along with a mixture comprising of benzene (156 g) and propylene (28 g). The reaction was carried out at 130° C. and 2758 kPa for 4 hours. The catalyst performance was assessed and shown in Table 1.

### EXAMPLE 8

The deactivated catalyst of Example 7 was regenerated in a flowing zone at 150° C. in a horizontal tub furnace for 16 hours using an ozone-containing gas having the ozone concentration of 1.5 wt.% in a flow rate of 3500 sccm (standard cubic centimeter, 20° C., 1 atmosphere). One-half gram of the regenerated catalyst was evaluated for benzene alkylation with propylene according to the method described in Example 1. The catalyst performance was assessed and shown in Table 1.

TABLE 1
Characterization of Spent and Regenrated Catalyst

| | Example 1 | Example 2 | Example 3 | Example 4 | Example 5 | Example 6 | Example 7 | Example 8 |
|---|---|---|---|---|---|---|---|---|
| | Spent | Regenerated | Regenerated | Regenerated | Regenerated | Regenerated | Regenerated | Regenerated |
| | Catalyst | Catalyst | Catalyst | Catalyst | Catalyst | Catalyst | Catalyst | Catalyst |
| Normalized | 100% | 202% | 202% | 183% | 100% | 167% | 100% | 240% |
| Cumene | | | | | | | | |
| Activity (to | | | | | | | | |
| 100% for Spent | | | | | | | | |
| Catalyst) (2^{nd} | | | | | | | | |
| Order Rate | | | | | | | | |
| Constant) | | | | | | | | |
| Normalized | 100% | 113% | 116% | 117% | 100% | 130% | 100% | 156% |
| DIPB/IPB | | | | | | | | |
| Selectivity (to | | | | | | | | |
| 100% for Spent | | | | | | | | |
| Catalyst) | | | | | | | | |
| Normalized Tri- | 100% | 134% | 141% | 146% | 100% | 185% | 100% | 222% |
| IPB/IPB | | | | | | | | |
| Selectivity (to | | | | | | | | |
| 100% for Spent | | | | | | | | |
| Catalyst) | | | | | | | | |

It will be seen from Table 1 that the regeneration method of the invention, in which the deactivated catalyst was regenerated using the ozone-containing gas flow at low temperature (Examples 3, 4, 6 and 8), is effective at restoring the activity and selectivity of the catalyst, and the restored activity and selectivity was comparable to a conventional high temperature N₂/O₂ calcination at high temperature 538° C. (Example 2).

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values take into account experimental error and variations that would be expected by a person having ordinary skill in the art.

The term “comprising” (and its grammatical variations) as used herein is used in the inclusive sense of “having” or “including” and not in the exclusive sense of “consisting only of” The terms “a” and “the” as used herein are understood to encompass the plural as well as the singular.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

The foregoing description of the disclosure illustrates and describes the present disclosure. Additionally, the disclosure shows and describes only the preferred embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

What is claimed is:

1. A process for alkylating an alkylatable aromatic compound comprising the step of contacting the alkylatable aromatic compound and an alkylating agent with a regenerated catalyst comprising a molecular sieve under alkylation conditions to form an alkylated aromatic compound, wherein the regenerated catalyst was regenerated by a method comprising the step of contacting an at least partially deactivated catalyst with an ozone-containing gas under regeneration conditions to produce the regenerated catalyst.

2. The process of claim 1, wherein the regeneration conditions comprise a temperature from about 50° C. to about 250° C.

3. (canceled)

4. The process of claim 1, wherein the regeneration conditions comprise a regeneration period from 10 min to about 48 hours.

5. The process of claim 1, wherein the regeneration conditions comprise a regeneration period from 10 hours to about 24 hours.

6. The process of claim 1, wherein the ozone-containing gas has an ozone concentration of from about 0.1 wt. % to about 10 wt. %.

7. The process of claim 1, wherein the ozone-containing gas has an ozone concentration of from about 0.5 to about 5 wt. %.

8. The process of claim 1, wherein the ozone-containing gas has a flow rate of about 0.1 to about 900 volumes of ozone-containing gas to catalyst volume per minute under the regeneration conditions.

9. The process of claim 1, wherein the molecular sieve is selected from the group consisting of a MCM-22 family molecular sieve, faujasite, mordenite, zeolite beta, and combinations thereof.

10. The process of claim 9, wherein the MCM-22 family molecular sieve is selected from the group consisting of MCM-22, PSH-3, SSZ-25, MCM-36, MCM-49, MCM-56, ERB-1, EMM-10, EMM-10-P, EMM-12, EMM-13, UZM-8, UZM-8HS, ITQ-1, ITQ-2, ITQ-30 and combinations thereof.

11. The process of claim 1, wherein the alkylation conditions are such that the alkylatable aromatic compound and the alkylating agent are in at least partial liquid phase.

12. The process of claim 1, wherein the alkylating agent comprises an olefinic group having 1 to 5 carbon atoms or a poly-alkylaromatic compound.

13. The process of claim 1, wherein the alkylating agent is ethylene or propylene.

14. The process of claim 1, wherein the alkylatable aromatic compound is benzene.

15. The process of claim 1, wherein alkylation conditions comprise a temperature of from 50° C. to about 400° C. and a pressure of from about 100 kPa to about 7000 kPa.

16. A method of regenerating an at least partially deactivated aromatic catalyst comprising a molecular sieve, the method comprising the step of contacting the deactivated alkylation catalyst with an ozone-containing gas under regeneration conditions.

17. The method of claim 16, wherein the regeneration conditions comprise a temperature from about 50° C. to about 250° C.

18. (canceled)

19. The method of claim 16, wherein the regeneration conditions comprise a regeneration period from 10 minutes to 48 hours.

20. (canceled)

21. The method of claim 16, wherein the ozone-containing gas has an ozone concentration of from about 0.1 to about 10 wt. %.

22. (canceled)

23. The method of claim 16, wherein the ozone-containing gas has a volumetric flow rate of about 0.1 to about 900 volumes of ozone-containing gas to catalyst volume per minute under the regeneration conditions.

24. The method of claim 16, wherein the molecular sieve of the alkylation catalyst or the transalkylation catalyst is selected from the group consisting of a MCM-22 family molecular sieve, faujasite, mordenite, zeolite beta, and combinations thereof.

25. The method of claim 24, wherein the MCM-22 family molecular sieve is selected from the group consisting of MCM-22, PSH-3, SSZ-25, MCM-36, MCM-49, MCM-56, ERB-1, EMM-10, EMM-10-P, EMM-12, EMM-13, UZM-8, UZM-8HS, ITQ-1, ITQ-2, ITQ-30 and combinations thereof.
